(19) United States
(12) Patent Application Publication
(10) Pub. No.: US 2019/0343442 A1
(43) Pub. Date: Nov. 14, 2019

(54) SYSTEM AND METHOD TO DETERMINE HEART RATE VARIABILITY COHERENCE INDEX

(71) Applicant: Hill-Rom Services PTE. LTD., Singapore (SG)

(72) Inventors: Aye AUNG, Singapore (SG); Yaolong LOU, Singapore (SG); Yuan Ing CHOW, Singapore (SG)

(21) Appl. No.: 16/353,138

(22) Filed: Mar. 14, 2019

Related U.S. Application Data
(60) Provisional application No. 62/669,444, filed on May 10, 2018.

(51) Int. Cl. A61B 5/16; A61B 5/024; A61B 5/00

(52) U.S. Cl. CPC A61B 5/165; A61B 5/02405; A61B 5/7225; A61B 5/7246; A61B 5/02416

(57) ABSTRACT

A stress level monitoring system includes a controller and a pulse oximeter electrically coupled to the controller. The pulse oximeter measures a photoplethysmograph signal. The controller determines a stress level of a patient from the photoplethysmograph signal.

### CROSS-REFERENCE TO RELATED APPLICATIONS

This application claims priority under 35 U.S.C. § 119(e) to U.S. Provisional Patent Application Ser. No. 62/669,444, filed May 10, 2018, and titled “SYSTEM AND METHOD TO DETERMINE HEART RATE VARIABILITY COHERENCE INDEX,” which is expressly incorporated by reference herein.

### BACKGROUND

The present disclosure relates to photoplethysmograph signals from a pulse oximeter and particularly, to a method for objectively determining patient stress from a photoplethysmograph signal.

Often, patients experience stress when in a caregiver facility or when receiving care at home. For example, the patient may experience stress due to medical procedures, medications, or length of stay at a facility, to name a few stress inducers. Generally, it is very subjective to determine a patient's stress level. However, to properly manage care, the caregiver must be aware of the patient's stress levels. It is desirable to determine a patient's stress level without subjecting the patient to additional tests, procedures, or medical equipment. That is, it is desirable to determine the patient's stress level with existing equipment that is being used to monitor the patient. It is also desirable to objectively quantify a level of the patient's stress. By quantifying stress, the caregiver may provide more accurate care to the patient.

### SUMMARY

The present disclosure includes one or more of the features recited in the appended claims and/or the following features which, alone or in any combination, may comprise patentable subject matter.

According to an aspect of the disclosed embodiments, a method of determining a stress level of a patient from a photoplethysmograph signal may include acquiring a photoplethysmograph signal from a pulse oximeter. The method may also include detecting peaks and valleys in the photoplethysmograph signal to determine a heart rate variability waveform. The method may also include detrending the heart rate variability waveform to determine a detrended waveform. The method may also include normalizing the detrended waveform to determine a normalized waveform. The method may also include determining a dominant frequency of the normalized waveform. The method may also include determining a coherence index of the heart rate variability waveform based on the dominant frequency.

It may be contemplated that the coherence index is between 0 and 1. A smoothness of the heart rate variability waveform may indicate a stress level of a patient.

In some embodiments, a dominant frequency within a range of 0.04 to 0.15 Hz may indicate a combination of sympathetic nervous system influence and parasympathetic nervous system influence. A dominant frequency within a range of 0.15 to 0.4 Hz may indicate parasympathetic nervous system activity. The coherence index may be a ratio of dominant frequencies within a range of 0.04 to 0.15 Hz to dominant frequencies within a range of 0.15 to 0.4 Hz.

Optionally, the method may include sampling at least one minute of the photoplethysmograph signal. The method may also include detecting peaks and valleys in the photoplethysmograph signal further comprises performing a peak to peak detection algorithm on a sample of the photoplethysmograph signal. The method may also include determining a coherence index further comprises deriving a normalized correlation index of the heart rate variability waveform with a sine waveform having the dominant frequency. The method may also include determining a coherence index further comprises multiplying the sine waveform having the dominant frequency with the heart rate variability waveform. The method may also include resampling the photoplethysmograph signal to acquire uniform heart rate variability waveforms.

According to another aspect of the disclosed embodiments, a stress level monitoring system may include a controller and a pulse oximeter electrically coupled to the controller. The pulse oximeter may measure a photoplethysmograph signal. The controller may determine a stress level of a patient from the photoplethysmograph signal by acquiring a photoplethysmograph signal from a pulse oximeter. The controller may also detect peaks and valleys in the photoplethysmograph signal to determine a heart rate variability waveform. The controller may also detrend the heart rate variability waveform to determine a detrended waveform. The controller may also normalize the detrended waveform to determine a normalized waveform. The controller may also determine a dominant frequency of the normalized waveform. The controller may also determine a coherence index of the heart rate variability waveform based on the dominant frequency.

It may be contemplated that the coherence index is between 0 and 1. A smoothness of the heart rate variability waveform may indicate a stress level of a patient.

In some embodiments, a dominant frequency within a range of 0.04 to 0.15 Hz may indicate a combination of sympathetic nervous system influence and parasympathetic nervous system influence. A dominant frequency within a range of 0.15 to 0.4 Hz may indicate parasympathetic nervous system activity. The coherence index may be a ratio of dominant frequencies within a range of 0.04 to 0.15 Hz to dominant frequencies within a range of 0.15 to 0.4 Hz.

Optionally, the controller may sample at least one minute of the photoplethysmograph signal. The controller may perform a peak to peak detection algorithm on a sample of the photoplethysmograph signal. The controller may derive a normalized correlation index of the heart rate variability waveform with a sine waveform having the dominant frequency. The controller may multiply the sine waveform having the dominant frequency with the heart rate variability waveform. The controller may resample the photoplethysmograph signal to acquire uniform heart rate variability waveforms.

According to yet another aspect of the disclosed embodiments, a method of determining a stress level of a patient from a photoplethysmograph signal includes acquiring a photoplethysmograph signal from a pulse oximeter. The method may also include calculating a heart rate waveform from the photoplethysmograph signal. The method may also include determining a dominant frequency of the heart rate waveform. The method may also include generating a sine wave from the dominant frequency. The method may also include determining a coherence index of the heart rate waveform by multiplying the heart rate waveform by the sine wave.

In some embodiments, the coherence index may be between 0 and 1. A smoothness of the heart rate waveform may indicate a stress level of a patient. A dominant frequency within a range of 0.04 to 0.15 Hz may indicate a combination of sympathetic nervous system influence and parasympathetic nervous system influence. A dominant frequency within a range of 0.15 to 0.4 Hz may indicate parasympathetic nervous system influence. The coherence index may be a ratio of dominant frequencies within a range of 0.04 to 0.15 Hz to dominant frequencies within a range of 0.15 to 0.4 Hz.

Optionally, the method includes sampling at least one minute of the photoplethysmograph signal. The method may also include resampling the photoplethysmograph signal to acquire uniform heart rate variability waveforms.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a pair of graphs illustrating heart rates of a patient, wherein a first graph illustrates the heart rate of a patient having high stress, and a second graph illustrates the heart rate of a patient having low stress;

FIG. 2 is a graph illustrating a Fast Fourier Transform of a patient's heart rate waveform; wherein the graph has numerous peaks indicative of sympathetic and parasympathetic nervous system influence on the heart rate;

FIG. 3 is a first graph of a photoplethysmograph signal, and a second graph of a peak to peaks detection of the photoplethysmograph signal;

FIG. 4 is a graph illustrating a peak and valley detection of a photoplethysmograph signal that is converted into a heart rate variation graph;

FIG. 5 is a sample heart rate waveform of a patient experiencing stress;

FIG. 6 is a flowchart of a method for objectively quantifying a patient's stress level from a photoplethysmograph signal;

FIG. 7 is a graph of a filtered photoplethysmograph signal that has undergone peak and valley detection to generate a heart rate variation graph;

FIG. 8 is a graph of a heart rate waveform and a Fast Fourier Transform of the heart rate waveform;

FIG. 9 is a graph of a heart rate waveform multiplied by a sine wave to determine an objective correlation index; and

FIG. 10 is a perspective view of a system for determining a patient's stress level from a photoplethysmograph signal.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

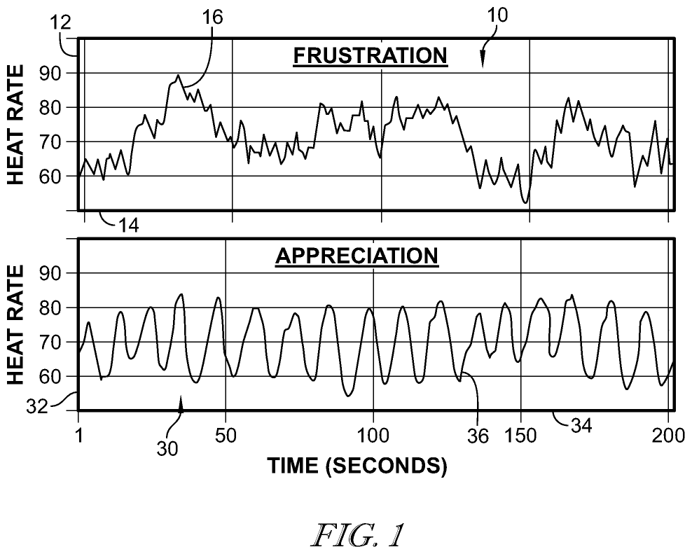

Generally, a patient's level of stress can be subjectively measured based on a heart rate variability of the patient. For example, referring to FIG. 1, a heart rate graph 10 is provided illustrating a patient that is generally frustrated or exposed to high levels of stress. The graph 10 illustrates a heart rate 12 of the patient on the y-axis and a time 14 on the x-axis of the graph 10. In the illustrative embodiment, the heart rate 12 is measured between 60 beats per minute and 90 beats per minute. The time 14 is measured over 200 seconds. A heart rate waveform 16 illustrates the patient's heart rate 12 over 200 seconds 14. As can be seen in the graph 10, the waveform 16 includes numerous jagged peaks and valleys. That is, the waveform 16 is not smooth. Generally, a waveform 16 that is not smooth is indicative of high stress in the patient.

Also seen in FIG. 1, a graph 30 illustrates a heart rate 32 on the y-axis over time 34 on the x-axis. In the illustrative graph 30, the heart rate 32 is illustrated between 60 beats per minute and 90 beats per minute. The time 34 is measured over 200 seconds. A waveform 36 illustrates the patient's heart rate 32 over 200 seconds 34. As can be seen in the graph 30, the waveform 36 is generally smooth and does not include jagged peaks and valleys. Generally, a waveform 36 that is smooth is indicative of low stress levels in the patient.

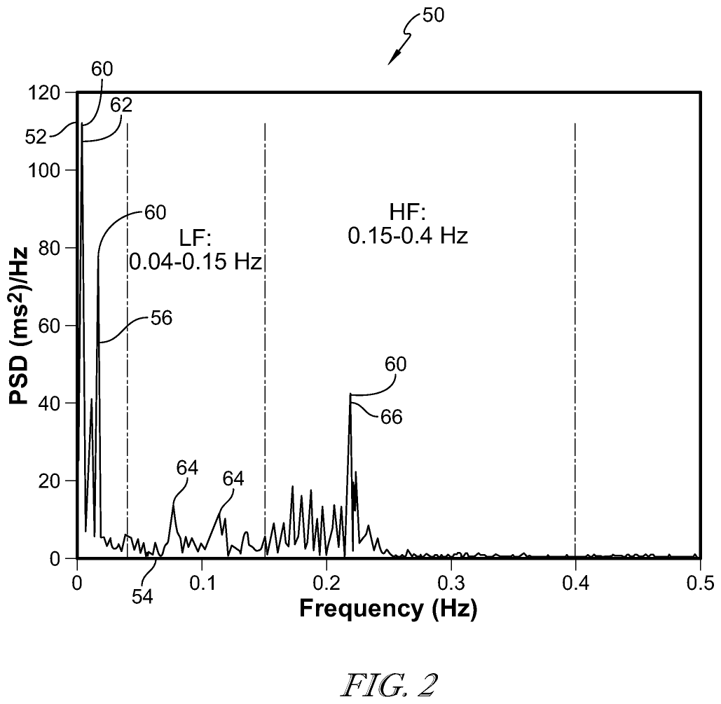

Further information regarding the patient's stress can be derived from a Fourier Transform of the heart rate waveform. For example, by taking a Fast Fourier Transform of the heart rate waveform, additional information regarding stress levels can be subjectively obtained. FIG. 2 illustrates a Fast Fourier Transform 50 of a patient's heart rate waveform. The Fast Fourier Transform 50 includes a y-axis 52 that measures time squared over frequency in Hertz. The Fast Fourier Transform 50 also includes an x-axis 54 that measures frequency in Hertz. Generally, a line 56 on the Fast Fourier Transform 50 includes numerous peaks 60. A first peak 62 appears at approximately 0 Hertz. Other peaks 64 occur between 0.04 Hz and 0.15 Hz. Generally, a peak that occurs between 0.04 Hz and 0.15 Hz indicates a combination of sympathetic nervous system influence and parasympathetic nervous system influence on the patient's heart rate. Additional peaks 66 occur between 0.15 Hz. and 0.4 Hz. Generally, a peak that occurs between 0.15 Hz. and 0.4 Hz indicates parasympathetic nervous system influence on the patient's heart rate.

Accordingly, based on the heart rate variability, as seen in FIG. 1, and the peaks of the Fast Fourier Transform, as seen in FIG. 2, a caregiver can subjectively assess a stress level of the patient. That is, a smoothness of the heart rate may generally indicate stress. Moreover, the peaks of the Fast Fourier Transform may indicate whether the sympathetic nervous system or the parasympathetic nervous system has contributed to the patient's heart rate. Unfortunately, assessments made from heart rate waveform and Fast Fourier Transforms are subjective and do not provide an objective assessment of the patient's stress level.

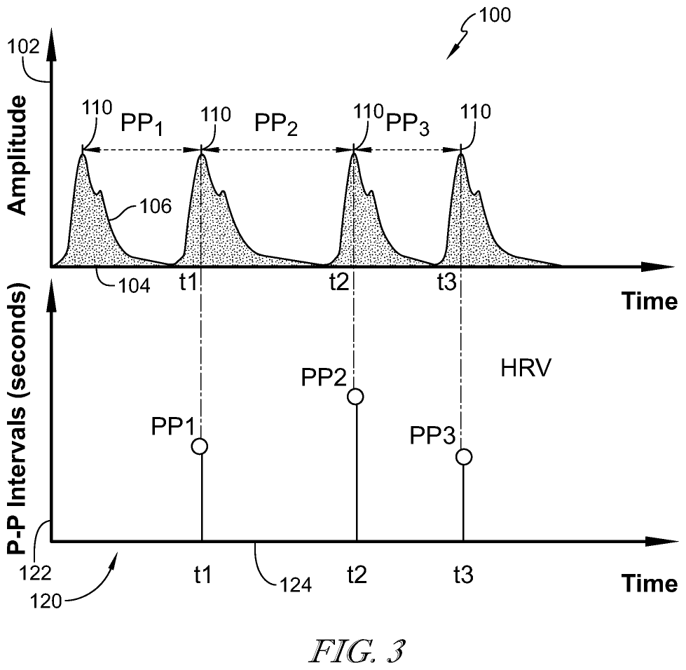

Referring to FIG. 3 a photoplethysmograph signal taken from a pulse oximeter can be manipulated to determine a heart rate variability. Accordingly, the patient's heart rate can be determined without having to use additional equipment in the healthcare facility, and a pulse oximeter alone can determine the patient's heart rate. The photoplethysmograph signal is shown in a graph 100 displaying amplitude 102 on the y-axis and time 104 on the x-axis. A line 106 displays the patient's oxygen levels as a function of amplitude 102 over time 104. The line 106 includes a plurality of peaks 110 and valleys 112. A second graph 120 illustrates peak to peak detection, as performed on the graph 100. The peak to peak detection 120 illustrates the peak to peak intervals 122 over time 124. In the exemplary embodiment, three peak to peak detections are illustrated on the graph 120. It should be noted that the number of peak to peak detections is dependent on a length of a time sample taken from the graph 100.

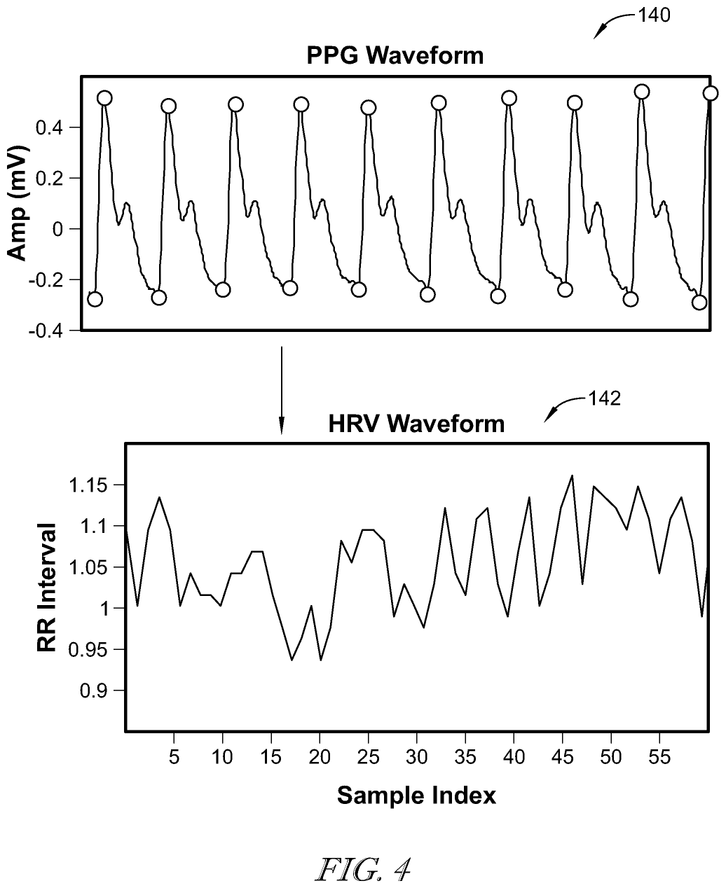

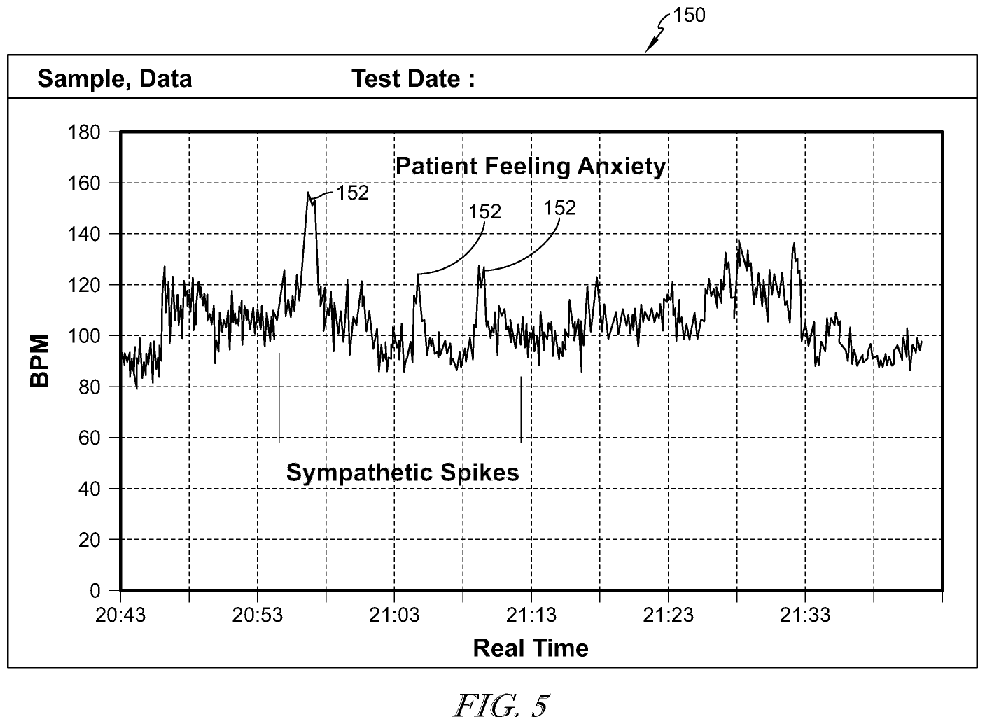

FIG. 4 illustrates a photoplethysmograph signal 140 converted to a heart rate variability waveform 142 using peak to peak detection. From the heart rate variability waveform 142 a general subjective assessment of the patient's stress levels can be determined. For example, the heart rate variability waveform 142 can be converted to a heart rate waveform 150, as illustrated in FIG. 5. The heart rate waveform of FIG. 5 may be indicative of a patient experiencing stress and frustration. As set forth above, the jagged peaks and valleys of the heart rate waveform 150 are indicative of high stress levels. Additionally, sympathetic spikes 152 in the heart rate waveform 150 also indicate general stress within the patient. Unfortunately, as set forth above, the determination of stress levels is still subjective and can't be quantified by the caregiver.

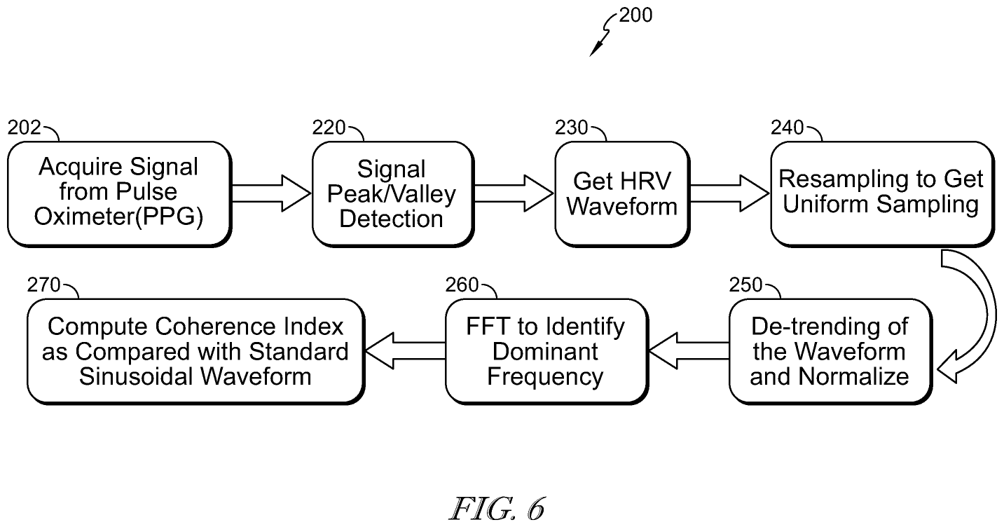

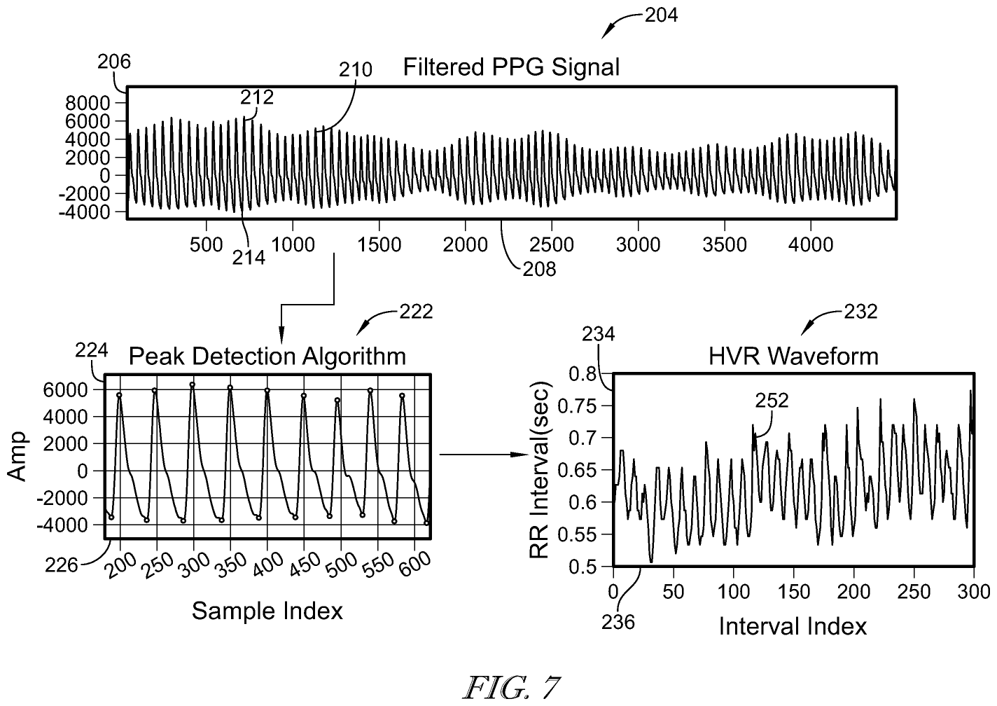

FIG. 6 illustrates a method 200 for objectively quantifying a patient's stress level using a photoplethysmograph signal. At block 202 a photoplethysmograph signal is acquired from a pulse oximeter. For example, the pulse oximeter is coupled to the patient's finger to retrieve a signal related to the patient's oxygen saturation in the blood. The photoplethysmograph signal is filtered, for example, using a band pass filter. An example of a filter photoplethysmograph signal is provided in the graph 204 in FIG. 7. The graph 204 includes a y-axis indicating an amplitude 206 of the signal and an x-axis indicating a sample index 208 of the signal. The line 210 shows the amplitude 206 as a function of sample index 208. The line 210 includes a series of peaks 212 and valley 214.

At block 220 of the method 200, peak and valley detection is performed on the filtered signal 210 to identify the peaks 212 and valleys 214 of the signal 210. The peaks 212 and valleys 214 can be graphed, as shown in the graph 222 of FIG. 7. The graph 222 includes amplitude 224 (y-axis) as a function of sample index 226 (x-axis). The graph 222 is utilized to derive the heart rate variation of the patient, at block 230, and as illustrated in graph 232 of FIG. 7. The graph 232 includes the respiratory rate interval 234 in seconds (y-axis) as a function of interval index 236 (x-axis). As set forth above, subjective determinations regarding the patient's stress level can be made from the graph 232. At block 240, the photoplethysmograph signal is resampled to achieve uniform sampling of the photoplethysmograph signal.

Once uniform sampling is achieved, at block 250, the waveform 252 of graph 232 is de-trended and normalized. That is, shifted and scaled versions of the data are created with the intention is that the normalized values allow the comparison of corresponding normalized values for different datasets in a way that eliminates the effects of certain gross influences, as in an anomaly time series. Some types of normalization involve only a rescaling to arrive at values relative to some size variable.

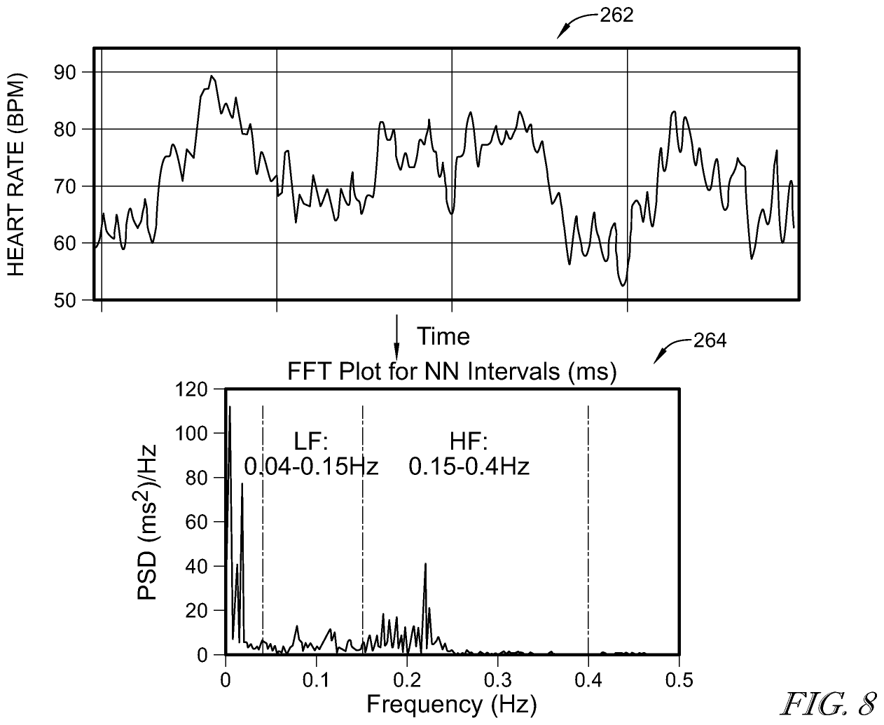

At block 260, the heart rate waveform 262, as illustrated in FIG. 8, is transformed with a Fast Fourier Transform. A Fast Fourier Transform 264 is also illustrated in FIG. 8. As described above, the dominant peaks in the Fast Fourier Transform 264 may be analyzed to determine subjective data about the patient's stress levels. To determine objective data about the patient's stress levels, the method 200 continues at block 270, where a coherence index is calculated.

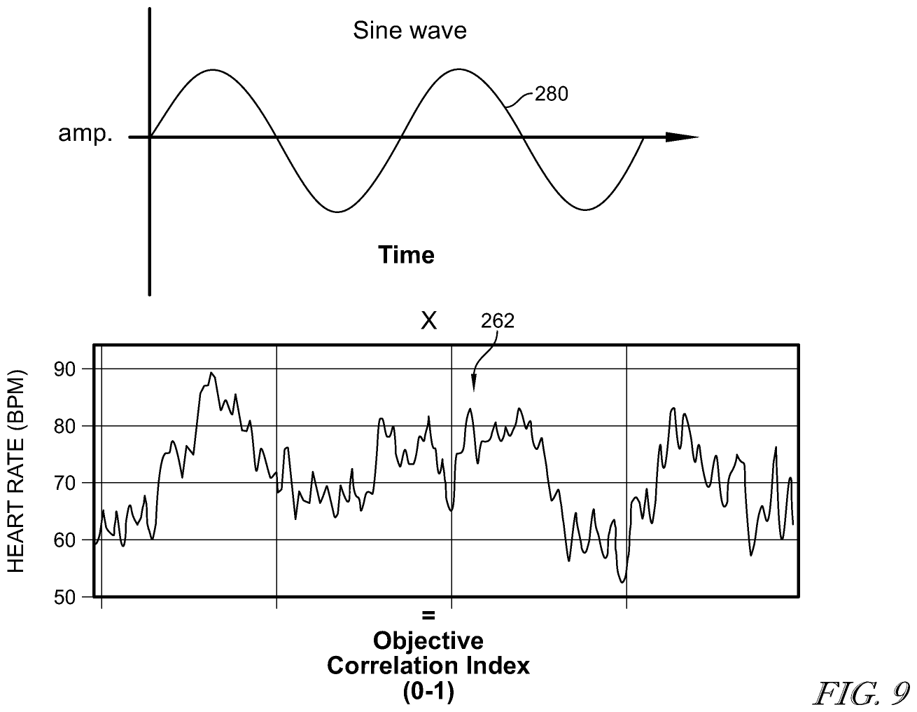

FIG. 9 graphically illustrates the method for determining the coherence index or objective correlation index. A sine wave is produced based on a peak frequency of the Fast Fourier Transform 264. For example, the Fast Fourier Transform 264 includes a peak frequency at 0.22 Hz. Accordingly, a sine wave 280 is generated with a frequency of 0.22 Hz. The sine wave 280 is multiplied by the heart rate waveform 262 to achieve a number between 0 and 1. The number between 0 and 1 provides a coherence index that objectively determines a patient's stress levels. For example, a number closer to 0 is indicative of a high stress level, whereas, a number closer to 1 is indicative of a low stress level in the patient. In some embodiments, the coherence index is a ratio of dominant frequencies within a range of 0.04 to 0.15 Hz to dominant frequencies within a range of 0.15 to 0.4 Hz.

Accordingly, a photoplethysmograph signal can be utilized to provide various information regarding the patient. First, the photoplethysmograph signal is indicative of the oxygen saturation of the patient. Second, the photoplethysmograph signal can be converted to determine a heart rate variation of the patient. Third, subjective information regarding stress levels can be derived from the heart rate waveform of the patient and a Fast Fourier Transform of the heart rate waveform. Lastly, a quantifiable objective score can be given to the patient's stress levels.

Heart rate variability is the patient's heart response to the central nervous system, which can measure the activation of the patient's autonomic nervous systems. Heart rate variability is a variation of heart beat-to-beat intervals, which can be extracted from a photoplethysmograph signal. The beat-to-beat intervals can be derived by using the peak detection algorithm. The coherence index is derived from the similarity of heart rate variability waveform with a pure sinusoidal wave by using a correlation analysis. The coherence index measures the heart rhythm coherence (sine wave-like rhythmic pattern) which implies the increased parasympathetic activity. The lower the index, the higher the stress condition is. As such, an objective score to provide the patient's stress condition is provided.

The system and method described herein require almost no additional when using an existing vital signs monitor and only additional software modification is required. The system and method described herein are non-invasive, portable, and suitable for homecare.

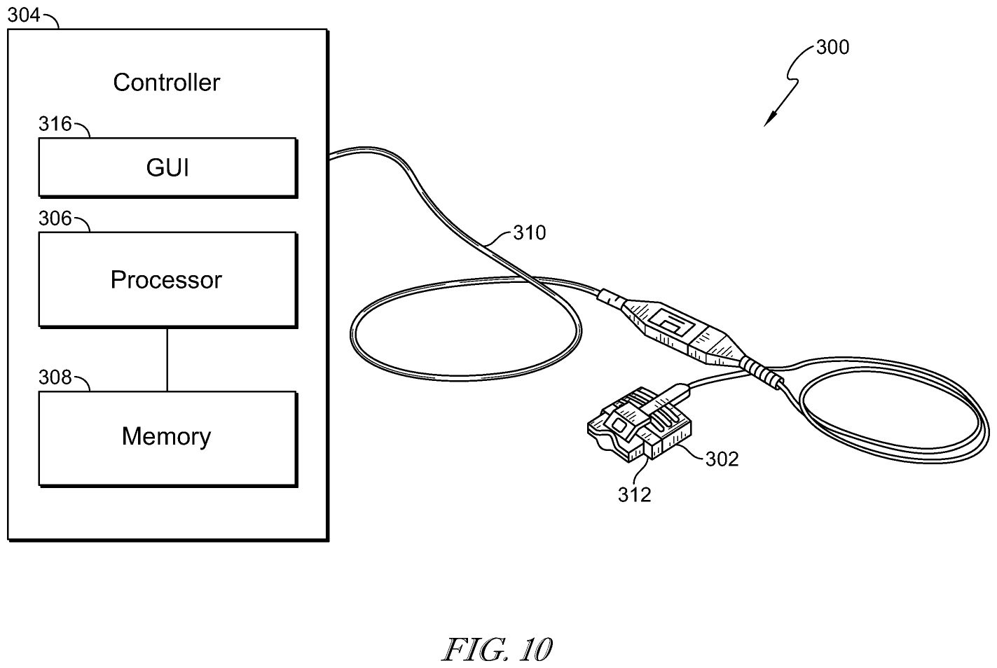

Referring to FIG. 10, a stress level monitoring system 300 includes a pulse oximeter 302 coupled to a controller 304. The controller 304 includes a processor 306 and a memory 308. The processor 306 may be embodied as any type of processor capable of performing the functions described herein. The processor 306 may be embodied as a dual-core processor, a multi-core or multi-threaded processor, digital signal processor, microcontroller, or other processor or processing/controlling circuit with multiple processor cores or other independent processing units. The memory 308 may be embodied as any type of volatile or non-volatile memory or data storage capable of performing the functions described herein. In operation, the memory 308 may store various data and software used during operation of the electronic controller 304 such as operating systems, applications, programs, libraries, and drivers. The memory 308 includes a plurality of instructions that, when read by the processor, cause the processor 306 to perform the functions described herein.

The pulse oximeter 302 is electrically coupled to the controller 304 via a cable 310. In some embodiments, the cable 310 includes a universal serial bus (USB) connector that is configured to connect to a USB port (not shown) provided on the controller 304. The pulse oximeter 302 provides a non-invasive method for monitoring a patient's oxygen saturation (SO₂) through a finger monitor 312 that is positioned on the patient's finger. In some embodiments, the pulse oximeter provides data related to the patient's peripheral oxygen saturation (SpO₂). In other embodiments, a monitor may be provided to measure the patient's arterial oxygen saturation (SaO₂) from arterial blood gas analysis. In some embodiments, the pulse oximeter 302 may be coupled to the patient's earlobe, foot, or any other thin part of the patient's body. The pulse oximeter 302 passes two wavelengths of light through the body part to a photodetector. The pulse oximeter 302 measures the changing absorbance at each of the wavelengths, allowing the pulse oximeter 302 to determine absorbency due to the pulsing arterial blood alone, excluding venous blood, skin, bone, muscle, and fat.

The pulse oximeter 302 is operable to detect data related to the patient's SpO₂ and heart rate. The pulse oximeter 302 also detects a photoplethysmograph signal (PPG) of the patient. The data acquired by the pulse oximeter 302 is transmitted to the controller 304. The controller 304 may display the data on the graphical user interface 316. The controller 304 is also operable to use the data to determine a heart rate variation waveform related to the patient's respiratory rate. While it may be known to acquire heart rate variation waveforms from raw PPG data, the methods described herein provide unique steps and data manipulation that are not currently applied to raw PPG data. As a result, the methods described herein represent an improvement over known methods for acquiring variation waveforms.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles can have been presented, they need not be utilized in combination, and many combinations of aspects and principles are possible in light of the various embodiments provided above.

1. A method of determining a stress level of a patient from a photoplethysmograph signal, the method comprising:
(i) acquiring a photoplethysmograph signal from a pulse oximeter;
(ii) detecting peaks and valleys in the photoplethysmograph signal to determine a heart rate variability waveform;
(iii) detrending the heart rate variability waveform to determine a detrended waveform;
(iv) normalizing the detrended waveform to determine a normalized waveform;
(v) determining a dominant frequency of the normalized waveform; and
(vi) determining a coherence index of the heart rate variability waveform based on the dominant frequency.

2. The method of claim 1, wherein the coherence index is between 0 and 1.

3. The method of claim 1, wherein a smoothness of the heart rate variability waveform indicates a stress level of a patient.

4. The method of claim 1, wherein a dominant frequency within a range of 0.04 to 0.15 Hz indicates a combination of sympathetic nervous system influence and parasympathetic nervous system influence.

5. The method of claim 4, wherein a dominant frequency within a range of 0.15 to 0.4 Hz indicates parasympathetic nervous system influence.

6. The method of claim 5, wherein the coherence index is a ratio of dominant frequencies within a range of 0.04 to 0.15 Hz to dominant frequencies within a range of 0.15 to 0.4 Hz.

7. The method of claim 1, further comprising sampling at least one minute of the photoplethysmograph signal.

8. The method of claim 1, wherein detecting peaks and valleys in the photoplethysmograph signal further comprises performing a peak to peak detection algorithm on a sample of the photoplethysmograph signal.

9. The method of claim 1, wherein determining a coherence index further comprises deriving a normalized correlation index of the heart rate variability waveform with a sine waveform having the dominant frequency.

10. The method of claim 9, wherein determining a coherence index further comprises multiplying the sine waveform having the dominant frequency with the heart rate variability waveform.

11. The method of claim 1, further comprising resampling the photoplethysmograph signal to acquire uniform heart rate variability waveforms.

12. A stress level monitoring system comprising:
a controller, and
a pulse oximeter electrically coupled to the controller, the pulse oximeter measuring a photoplethysmograph signal,
wherein the controller determines a stress level of a patient from the photoplethysmograph signal by:
(i) acquiring a photoplethysmograph signal from a pulse oximeter;
(ii) detecting peaks and valleys in the photoplethysmograph signal to determine a heart rate variability waveform;
(iii) detrending the heart rate variability waveform to determine a detrended waveform;
(iv) normalizing the detrended waveform to determine a normalized waveform;
(v) determining a dominant frequency of the normalized waveform; and
(vi) determining a coherence index of the heart rate variability waveform based on the dominant frequency.

13. The system of claim 12, wherein the coherence index is between 0 and 1.

14. The system of claim 12, wherein a smoothness of the heart rate variability waveform indicates a stress level of a patient.

15. The system of claim 12, wherein a dominant frequency within a range of 0.04 to 0.15 Hz indicates a combination of sympathetic nervous system influence and parasympathetic nervous system influence.

16. The system of claim 15, wherein a dominant frequency within a range of 0.15 to 0.4 Hz indicates parasympathetic nervous system activity.

17. The system of claim 16, wherein the coherence index is a ratio of dominant frequencies within a range of 0.04 to 0.15 Hz to dominant frequencies within a range of 0.15 to 0.4 Hz.

18. The system of claim 12, wherein the controller samples at least one minute of the photoplethysmograph signal.

19. The system of claim 12, wherein the controller performs a peak to peak detection algorithm on a sample of the photoplethysmograph signal.

20. The system of claim 12, wherein the controller derives a normalized correlation index of the heart rate variability waveform with a sine waveform having the dominant frequency.

21. The system of claim 20, wherein the controller multiplies the sine waveform having the dominant frequency with the heart rate variability waveform.

22. The system of claim 12, wherein the controller resamples the photoplethysmograph signal to acquire uniform heart rate variability waveforms.
